(19) United States
(12) Patent Application Publication
(10) Pub. No.: US 2003/0073136 A1
(43) Pub. Date: Apr. 17, 2003

(54) Method of and apparatus for performing flow cytometric measurements

(76) Inventors: Gerd Luedke, Holzgerlingen (DE); Bernd Nawracala, Karlsruhe (DE)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/271,535

(22) Filed: Oct. 17, 2002

(30) Foreign Application Priority Data
Oct. 17, 2001 (EP) 01 124764.0

(51) Int. Cl. G01N 33/53; G01N 33/567; G06F 19/00; G01N 33/48; G01N 33/50

(52) U.S. Cl. 435/7.2; 702/21

(57) ABSTRACT

Analyte cells with different properties flow through a flow cytometric detector arrangement. The number of analyte cells having the different properties is determined. To compensate for errors due to two or more analyte cells being simultaneously in the cytometric cell, the actual number of analyte cells with a specific property is calculated from the measured values of cells with this specific property by using a statistical model about the probability of simultaneous occurrences of cells in the cytometric cell. A data processing arrangement has a memory including the model and receives as input data the measured analyte cell numbers and uses an experimentally determined coincidence rate for calculating actual analyte cell numbers.

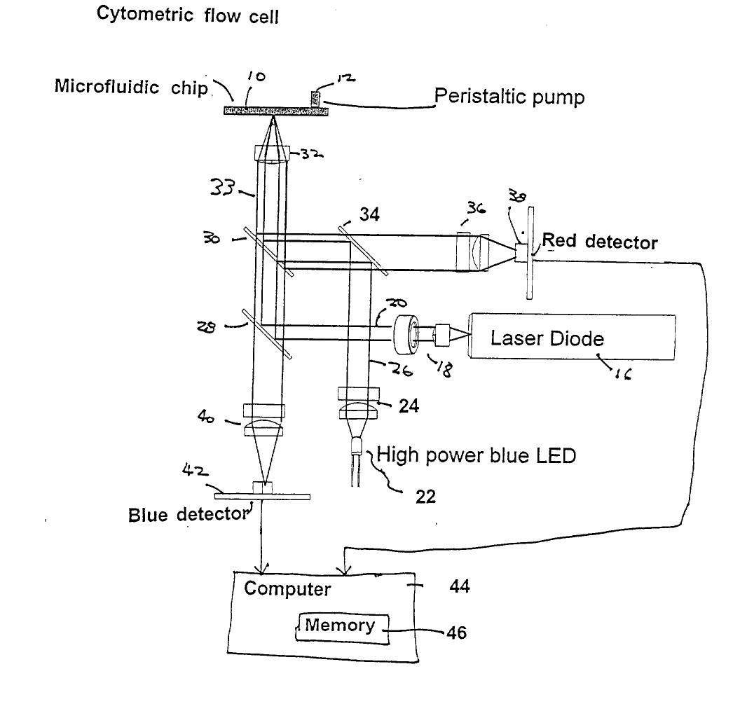

### FIELD OF THE INVENTION

The invention relates to a method of and apparatus for performing flow cytometric measurements and more particularly to such a method and apparatus wherein cells having different properties move through a detector arrangement.

### BACKGROUND OF THE INVENTION

Flow cytometry is a technique of measuring the properties of biological, analyte cells suspended in a fluid stream. The analyte cells are typically analyzed by fluorescence measurements, using, for example, a laser for exciting fluorescence radiation in the cells and an optical system with an optical detector to detect the emitted fluorescence radiation. In order to improve the analysis of specific biological properties of the analyte cell, it is customary to employ staining procedures to stain the analyte cell differentially according to the biological parameter of interest. The parameters of interest include staining for DNA content, viability, presence of extracellular or intracellular antigens, or if the analyte cell is undergoing apoptosis. The staining procedures enable the analyte cells to be interrogated individually for biological information. The thus obtained information can be used to identify different type analyte cells, and to separate them from each other and/or to sort them. A method of performing flow cytometric measurements is known, for example, from U.S. Pat. Nos. 5,466,572 and 5,747,349, incorporated by reference herein. The disclosure of the '572 patent indicates hematopoietec cell populations are separated to provide analyte cell sets and subsets as viable analyte cells and wherein high flow speeds of the analyte cell sets in a fluidic path reduce sorting time.

Since analyte cells are often heterogeneous within a certain population, it is impossible to study only a few analyte cells to obtain statistically relevant information about the analyte cell population. Consequently, several hundred or thousand analyte cells have to be measured to obtain relevant information. In practice, at least two, ideally independent, parameters are measured, so that the accuracy of the measurement is highly dependent on the ability of the flow cytometric apparatus to measure single cells individually.

Several construction principles for the fluidic path have been in use to achieve the goal of measuring analyte cells individually. According to a first construction principle, the analyte cells are injected by an injector tip into a fluid stream flowing around the tip in the direction of the tip axis. The fluid stream carries away the analyte cells in a narrow stream, and the detection is performed on this narrow stream of cells. In another example the fluid containing the cells is ejected from a nozzle having a very small orifice. The cells leaving the nozzle in a jet are detected by a suitable detector. In a third example, typically implemented on a microfluidic chip, the analyte cells are transported along a main channel and a buffer solution is introduced through a side channel, disposed at an acute angle relative to the main channel. At the intersection of the buffer solution stream and the analyte cells, a constriction in the flow of the cells occurs. Detection is performed at this constriction. In all of these arrangements, the analyte cells flow through a cytometric flow cell where the analyte cells are irradiated by a beam from the laser or some other optical source, such as a light emitting diode (LED). The fluid flow paths are ideally such that only one cell at a given point in time is in the cytometric flow cell and field of view of the detector.

Besides appropriate construction of the fluidic system, some other measures are known to attempt to cause only one analyte cell at a given point in time to be in the cytometric flow cell and detector field of view. Such measures include:

(1) using a low analyte cell concentration,

(2) making the detection spot very small,

(3) employing a high measurement frequency and a high data rate,

(4) analyzing the peak shapes and rejecting coincidence events.

The known methods, however, have certain deficiencies. With some of the methods, it is not always possible to ensure single analyte cell detection. Other methods require comparatively complex technical equipment and are therefore costly. None of the known methods appears to be able to reliably exclude analyte cell coincidences, i.e., the simultaneous occurrence of plural analyte cells in the cytometric flow cell and the detector field of view; the coincidences only become more improbable. Also, the method of peak rejection does not always work reliably.

It is thus an object of the invention to provide a new and improved method of and apparatus for performing flow cytometric measurements, wherein the number of analyte cells having different properties and flowing through a cytometric flow cell of a detector arrangement is determined.

Another object is to provide a new and improved method of and apparatus for performing flow cytometric measurements, wherein the above mentioned problem of simultaneous detection of several analyte cells in an easy, reliable manner is resolved with no additional instrumental expenditure.

### SUMMARY OF THE INVENTION

One aspect of the invention is related to a method of performing flow cytometric measurements wherein analyte cells with different properties are flowing through a flow cytometric cell of a detector arrangement, and wherein the number of analyte cells having specific different properties is determined. The method comprises compensating for errors due to two or more analyte cells being simultaneously detected as being in the flow cytometric cell. The compensation is performed by calculating the actual numbers of analyte cells with a specific property from measured values of the number of analyte cells with the specific property. The calculation is performed by using a statistical model about the probability of simultaneous occurrences of analyte cells being in the flow cytometric cell and detected by the detector arrangement.

Another aspect of the invention concerns an apparatus for performing flow cytometric measurements. The apparatus comprises a flow cytometric cell of a detector arrangement. The flow cytometric cell is arranged to be responsive to flowing analyte cells having different properties. A processor arrangement is coupled with the detector arrangement for (1) determining the number of analyte cells having each of the properties, and (2) compensating for errors due to two or more analyte cells being detected as simultaneously in the flow cytometric cell. The processor arrangement (1) performs the compensation by calculating the actual numbers of analyte cells with a specific property, and (2) includes a statistical model about the probability of analyte cells being detected as being simultaneously in the flow cytometric cell. The statistical model is part of the arrangement for calculating the actual numbers of analyte cells with the specific property.

A further aspect of the invention relates to a memory for use in a computer responsive to flow cytometric measurement, derived from a detector arrangement arranged to be responsive to analyte cells flowing through a flow cytometric cell, wherein the analyte cells have different properties. The memory stores a program for enabling the computer to (a) determine the numbers of analyte cells having each of the properties, and (b) compensate for errors due two or more analyte cells being detected as being simultaneously in the flow cytometric cell by calculating the actual numbers of analyte cells with a specific property. The program includes a statistical model about the probability of simultaneous occurrences of analyte cells being detected as being in the flow cytometric cell. The statistical model is part of the program for calculating the actual numbers of analyte cells with the specific property.

The present invention does not employ the prior art approach of dealing with the simultaneous occurrence of two or more analyte cells with a specific property at the flow cytometric cell of the detector by trying to ensure that only one analyte cell at a time is detected as being present at the flow cytometric cell. Rather, in the present invention there can be simultaneous occurrences of two or more cells with a specific property in the flow cytometric cell and detected by the optical detector. The present invention is based on the realization that errors caused by the simultaneous occurrences follow certain statistical regularities and can thus be compensated by using an appropriate data processing algorithm. An input parameter for the statistical model is the coincidence rate or probability of simultaneous occurrence of several analyte cells being in the flow cytometric cell and detected by the optical detector. Preferably, this coincidence rate is experimentally determined for each specific flow cytometer.

In an embodiment of the invention, the statistical model used for the error compensation is based on the assumption that the detection of analyte cells by the optical detector follows a Poisson distribution and that there is only simultaneous occurrence of two cells, but not of three or more cells. It has been found that these assumptions describe typical practical situations very well and lead to reliable measuring results.

Preferably, the statistical model also assumes that the probability of simultaneous occurrence of analyte cells in the flow cytometric cell is not dependent on the specific properties of the particular cells. Such properties might be, for example, different colors that have stained the analyte cells. The different colors are detected, for example, by a fluorescence detector. According to another preferred embodiment, the statistical model is based on the assumption that the signals corresponding to the specific properties of the analyte cells measured by the optical detector are additive.

The above described assumptions for the statistical model can be further refined by taking into account coincidences of three or more analyte cells. The coincidence rates are experimentally determined and are used as input parameters in the statistical model.

In the following, some embodiments of the invention are explained in more detail.

### BRIEF DESCRIPTION OF THE DRAWING

The FIGURE is a schematic diagram of a cytometric measuring apparatus incorporating a preferred embodiment of the present invention.

### DETAILED DESCRIPTION OF THE DRAWING

Reference is now made to the FIGURE wherein a cytometric flow cell 10, in the form of a microfluidic chip, preferably a microfluidic chip commercially available as a LAB CHIP, includes a carrier fluid containing biological analyte cells. Peristaltic pump 12 supplies pressure to the fluid to move the fluid in channels in cell 10. Different biological analyte cells in cytometric flow cell 10 have specific properties, typically being stained in various colors, such as bright red, faint red, bright blue and faint blue.

Laser diode 16 excites the biological analyte cells in cytometric flow cell 10 into a fluorescent state. To this end, laser diode 16 emits a coherent optical beam that is directed to the biological analyte cells in cytometric flow cell 10 by an optical system including lens arrangement 18 which produces a collimated optical beam 20. The biological analyte cells in cytometric flow cell 10 are also irradiated by optical energy produced by high power blue light emitting diode (LED) 22, the output of which is collimated by lens arrangement 24 into beam 26. Beam 20 is incident on semitransparent mirror 28 which direct beam 20 via semitransparent mirror 30 onto the analyte cells in cytometric flow cell 10 by way of microscope objective 32. Semitransparent mirrors 34 and 30 direct the optical energy from LED 22 onto microscope objective 32 which in turn directs the energy from the LED onto the biological analyte cells in cytometric flow cell 10. The stained cells fluoresce in response to the optical energy incident thereon to produce multicolor optical energy that is collimated by microscope objective 32 into collimated beam 33.

Mirror 30 reflects the red energy in beam 33 to red optical detector 38 via semitransparent mirror 34 and shaping lens 36. Semitransparent mirror 28 passes the blue energy in beam 33 to beam shaping lens arrangement 40, which directs the blue energy onto blue detector 42. Detectors 38 and 42 derive electric output signals representing the intensity of the red and blue optical energy respectively incident thereon. The electrical output signals of detectors 38 and 42 are coupled as inputs to computer 44 that includes memory 46 and is part of a data processing system having the usual input devices, such as a keyboard, and an output device, such as a computer monitor.

Typically, the microfluidic chip included in cytometric flow cell 10 includes a main channel that is about 75 micrometers in width and which is responsive to the biological analyte cells and the carrier fluid. A buffer solution is introduced through a side channel, disposed at an acute angle relative to the main channel. The fluids flowing through the side channel and the fluid flowing in the main channel are combined at the intersection of the main and side channels and flow together through the main channel. The intersection forms a detection region that is irradiated by the optical energy that microscope objective 32 directs onto cytometric flow cell 10. The portion of the main channel downstream of the intersection of the main channel and the side channel has a width of approximately 75 micrometers, the same width as the side channel. It is to be understood that the principles of the invention are not limited to cytometric flow cells that are configured as microfluidic chips although the invention has its greatest advantage with such cells.

According to a first embodiment of the invention computer 44 includes a memory 46 programmed to include a statistical model for correcting for coincidences of stained biological analyte cells that are in cytometric flow cell 10 and irradiated by the output beam of objective 32. The model takes into account cell doublets, i.e. two cells being simultaneously in cytometric flow cell 10 and detected by detectors 38 and 42, but does not consider triplets or higher cell aggregates.

The detection by detectors 38 and 42 of an analyte cell in flow cytometry is a statistically rare event. The probability of detectors 38 and 42 detecting such an event in a given time follows a Poisson distribution, not a binomial distribution. It is proper to assume that the events optical detectors 38 and 42 detect are statistically independent and occur with a probability Q, where Q is much smaller than 1. Such an assumption enables one to predict the probability of detectors 38 and 42 detecting a coincidence event as a function of Q. If n is the number of analyte cells simultaneously flow cytometric cell 14 and in the spot of detectors 38 and 42, i.e., in the field view of detectors 38 and 42, of the flow cytometer, the probability of detectors 38 and 42 viewing two cells simultaneously is:

Q(2)=Q²

For higher order coincidence events:

Q(n)=Qⁿ

The occurrence of coincidence events is a consequence of the Poisson distribution and contributes to the overall result of the measurement resulting from the outputs of detectors 38 and 42.

The coincidence rate p (=Q²) is experimentally measurable as is described below in more detail.

The effects of these coincidence events are of a statistical nature and are corrected with the help of the mathematical computer program model that computer 44 stores.

The following mathematical model, which memory 46 of computer 44 stores, considers cell doublets and is described in detail. The following terminology is used.

The capital letters A, B, C, D denote the measured fractions of stained biological analyte cells flowing through cytometric flow cell 10 and detected by detectors 38 and 42. The stained cells have different properties, such as different staining colors (e.g. red, blue, etc.). Each of these measured fractions is the ratio of the number of analyte cells having the specific property to the total number of analyte cells detected within a unit time.

The lower case letters a, b, c, d denote the real fractions of analyte cells of the different properties, respectively, as the analyte cells would be if there were no coincidences.

The letter p denotes the probability for two of the stained biological analyte cells in cytometric flow cell 10 being simultaneously irradiated by the output beam of objective 32; such stained cells are sometimes referred to as doublets herein.

In a specific example, the biological analyte cells are stained blue and red and distinctions are made between stained cells that are faint red, bright red, faint blue and bright blue. According to this example, the lower case letters a, b, c, d mean the following:

a: faint blue/faint red, i.e., the fraction of cells that are faint blue and faint red;

b: bright blue/faint red, i.e., the fraction of cells that are bright blue and faint red;

c: faint blue/bright red, i.e., the fraction of cells that are faint blue and bright red; and

d: bright blue/bright red, i.e., the fraction of cells that are bright blue and bright red.

The measured fraction, A, of stained analyte cells with a first property is the real fraction “a” times the probability (1−p) that the analyte cells do not form doublets, plus the real fraction of “a” times the probability that doublets of “a” are formed. The other fractions are formed analogously by adding the probability for singlets plus the probability for doublets. As a result, the following relations apply:

A=(1−p)a+p(a²)

B=(1−p)b+p(b²+2ab)

C=(1−p)c+p(c²+2ac)

D=(1−p)d+p(d²+2ad+2bd+2cd+2bc)

Solving these quadratic equations for the small letters a, b, c, d, yields the following results:

$$\begin{matrix}a = - \frac{( 1 - p )}{2 p} + \sqrt{{[ \frac{( 1 - p )}{2 p} ]}^{2} + \frac{A}{p}} \\ b = - \frac{( 1 - p ) + 2 ap}{2 p} + \sqrt{{[ \frac{( 1 - p ) + 2 ap}{2 p} ]}^{2} + \frac{B}{p}} \\ c = - \frac{( 1 - p ) + 2 ap}{2 p} + \sqrt{{[ \frac{( 1 - p ) + 2 ap}{2 p} ]}^{2} + \frac{C}{p}} \\ d = - \frac{( 1 - p ) + 2 ap + 2 bp + 2 cp}{2 p} + \\ \sqrt{{[ \frac{( 1 - p ) + 2 ap + 2 bp + 2 cp}{2 p} ]}^{2} + \frac{D}{p} - 2 bc}\end{matrix}$$

In that way, one can calculate the actual (real) values of “a,” “b,” “c,” and “d” from the measured values of A, B, C and D. It is only required to know the coincidence rate p, which can be determined experimentally. Typically, the coincidence rate p is measured by using a 1:1 mixture of red and blue stained analyte cells or beads. If this mixture is measured, a coincidence event results in an event that is simultaneously stained red and blue. The coincidence rate p for the generation of doublets is calculated from the measured data by using the following formula:

p=2*(number of coincidence events/number of all events)

Besides the experimental determination of the analyte cell coincidence rate, the coincidence rate can be calculated, if the probability for the occurrence of a single event is known. Under this condition, computer 44 calculates the overall coincidence rate with red and blue cells or beads as follows:

Coincidence rate=p²_{blue}+2*p_{blue}*p_{red}+p²_{red}

Computer 44 could calculate the coincidence rate of triplets or multiplets in an analogous way.

According to a practical example, coincidence rates for doublets and higher order multiplets can exceed 10% and even become 20% if analyte cell densities are high.

The above described computational steps are typically performed with the help of the data processing arrangement including computer 44 which is connected to be responsive to the flow through cytometer cell 10. The computer program of computer 44 receives as inputs from detectors 38 and 42 the number of (1) total events, (2) faint blue and faint red events, (3) faint blue and bright red events, (4) bright blue and faint red events, and (5) double bright events, i.e., bright red and bright blue. Using the experimentally determined value for p computer 44 calculates corrected values and displays the corrected values to a user.

The assumptions in the model stored in the memory 46 of computer 44 and used in the above embodiment are now described in more detail.

1. There are only analyte cell doublets. Triplets or higher aggregates of analyte cells do not occur.

2. The model only corrects for coincidence events. Physical doublets that are attached to each other do not occur.

3. Doublets are additive. This means that the coincidence of a faint and a bright event results in a bright event and not a faint event. This means that the signal of both events reach detectors 38 and 42. Computer 44 responds to the signals detectors 38 and 44 derive to time integrate the signals and combine the integrated signals into one signal.

4. The probability for coincidence is not dependent on the staining of the event. This means that the probability for two stained biological red analyte cells to generate a coincidence event is not different from the probability for two stained biological blue analyte cells generating a coincidence event, if both stained biological analyte cell kinds have the same occurrence probability.

These assumptions are true for the following conditions. Assumptions 1 and 2 are limitations of the model memory 46 of computer 44 stores. If one wants to correct triplets or higher order multiplets, the model memory 46 stores must be expanded. However, because of the statistical occurrence of the coincidence, this would be straight forward. The possible error that is introduced by a triplet is well below the error introduced by a doublet, because of the rare occurrence of triplets. If the probability for the occurrence of an event is p, the probability of the occurrence of an n-let is pⁿ.

Assumption 3 is true for all systems where the radiation emitted by one particle is not absorbed or reflected or quenched by another particle. This is especially true for biological systems which are very transparent.

Assumption 4 is generally true for all systems that are not modified in a way that the particles will have different affinities to each other. This means that if a blue stained analyte cell is carrying a receptor for a ligand on a red stained cell, conditions can arise where the probability for a red-blue doublet is higher than for a red-red doublet, even if both analyte cells have the same concentration. This, however, would be a physical doublet that is not corrected by the model (see assumption 2).

Generally, it can be said that the mentioned assumptions are reasonable for typical flow cytometric applications. It is understood, however, that in situations or with flow cytometers wherein the analyte cells have a behavior differing from the above, a modified statistical model might be used.

The application of the model is not restricted to fluorescence detection, but is applicable to all detection methods wherein the signals resulting from detection are integrated over a certain time, unless the signal of a coincidence event does not exceed the dynamic range of the detector and the signal of a single event is already detectable by the detector. Therefore, the method and apparatus can be used for optical detectors regardless of wavelength, for electrical detectors or detectors using radioactivity and all other detection methods and apparatus wherein the signals are integrated over a certain time.

1. A method of performing flow cytometric measurements wherein analyte cells with different properties are flowing through a flow cytometric cell of a detector arrangement, and wherein the number of analyte cells having specific different properties is determined, comprising compensating for errors due to two or more analyte cells being simultaneously detected as being in the flow cytometric cell by calculating the actual number of analyte cells with a specific property from measured values of the number of analyte cells with the specific property, the calculation being performed by using a statistical model about the probability of simultaneous occurrences of analyte cells being detected by the detector arrangement.

2. Method as in claim 1, wherein the statistical model is based on an assumption that there is only simultaneous occurrence of two analyte cells, but not of three or more analyte cells.

3. Method as in claim 2, wherein the statistical model is based on an assumption that the probability of simultaneous occurrence of the analyte cells is not dependent on the specific properties of the analyte cells.

4. Method as in claim 3, wherein the statistical model is based on an assumption that the signals corresponding to the specific properties of the analyte cells measured by the detector arrangement are additive.

5. Method as in claim 1, wherein the statistical model is based on an assumption that the probability of simultaneous occurrence of the analyte cells is not dependent on the specific properties of the analyte cells.

6. Method as in claim 1, wherein the statistical model is based on an assumption that the signals corresponding to the specific properties of the analyte cells measured by the detector arrangement are additive.

7. Method of claim 1, wherein the specific properties of the analyte cells are stainings with different colors.

8. Method as in claim 1, wherein the statistical model is based on the assumption that the detection of analyte cells by the detector arrangement follows a Poisson distribution.

9. Method as in claim 8, comprising the following steps:
(a) measuring the fractions (A, B, C, D) of analyte cells having specific properties, respectively,
(b) determining the real fractions (a, b, c, d) of analyte cells having said specific properties, using the experimentally determined probability (p) for simultaneous detection of plural analyte cells.

10. Method as in claim 2, comprising the following steps:
(a) measuring the fractions (A, B, C, D) of analyte cells having specific properties, respectively,
(b) determining the real fractions (a, b, c, d) of analyte cells having said specific properties, using the experimentally determined probability (p) for simultaneous detection of plural analyte cells.

11. Method as in claim 5, comprising the following steps:
(a) measuring the fractions (A, B, C, D) of analyte cells having specific properties, respectively,
(b) determining the real fractions (a, b, c, d) of analyte cells having said specific properties, using the experimentally determined probability (p) for simultaneous detection of plural analyte cells.

12. Method as in claim 6, comprising the following steps:
(a) measuring the fractions (A, B, C, D) of analyte cells having specific properties, respectively,
(b) determining the real fractions (a, b, c, d) of analyte cells having said specific properties, using the experimentally determined probability (p) for simultaneous detection of plural analyte cells.

13. Method as in claim 9 wherein the determining step is performed with the help of a data processing arrangement.

14. An apparatus for performing flow cytometric measurements, comprising a flow cytometric cell of a detector arrangement, the flow cytometric cell being arranged to be responsive to flowing analyte cells having different properties, a processor arrangement coupled with the detector arrangement for (a) determining the numbers of analyte cells having each of the properties, and (b) compensating for errors due to two or more analyte cells being detected as simultaneously in the flow cytometric cell, the processor arrangement being arranged for performing the compensation by calculating the actual numbers of analyte cells with a specific property, the data processor arrangement including a statistical model about the probability of simultaneous occurrences of analyte cells being detected as being simultaneously in the flow cytometric cell, the statistical model being part of the arrangement for calculating the actual numbers of analyte cells with the specific property.

15. Apparatus as in claim 14, wherein the detector arrangement is a fluorescence detector.

16. A memory for use in a computer adapted to be responsive to flow cytometric measurements, derived from a detector arrangement arranged to be responsive to analyte cells flowing through a flow cytometric cell, wherein the analyte cells have different properties, the memory storing a program for enabling the computer to (a) determine the numbers of analyte cells having each of the properties, (b) compensate for errors due to two or more analyte cells being detected as simultaneously in the flow cytometric cell, and (c) perform the compensation by calculating the actual numbers of analyte cells with a specific property; the program including a statistical model about the probability of simultaneous occurrences of analyte cells being detected as being simultaneously in the flow cytometric cell, the statistical model being arranged to assist the computer to calculate the actual numbers of analyte cells with specific property.

17. The memory of claim 16, wherein the statistical model is based on an assumption that only two analyte cells, but not three or more cells can be simultaneously in the flow cytometric cell.

18. The memory of claim 16, wherein the statistical model is based on an assumption that the probability of the analyte cells being simultaneously in the flow cytometric cell is not dependent on the specific properties of the analyte cells.

19. The memory of claim 17 wherein the memory is programmed to enable the computer to determine (a) the fractions (A, B, C, D) of analyte cells having specific properties, respectively, and (b) the real fractions (a, b, c, d) of analyte cells having said specific properties, using the experimentally determined probability (p) for plural analyte cells being simultaneously in the flow cytometric cell.

20. The memory of claim 17 wherein the statistical model is based on the assumption that the detection of stained cells by the detector arrangement follows a Poisson distribution.
